(19) United States
(12) Patent Application Publication
(10) Pub. No.: US 2016/0054306 A1
(43) Pub. Date: Feb. 25, 2016

(54) The cause and possible treatment of thyroid anti-body disease

(71) Applicant: Iryna Borisovna Shevchenko, Stockton, CA (US)

(72) Inventor: Iryna Borisovna Shevchenko, Stockton, CA (US)

(21) Appl. No.: 14/463,194

(22) Filed: Aug. 19, 2014

(51) Int. Cl. G01N 33/50

(52) U.S. Cl. CPC G01N 33/5091; G01N 2333/575; G01N 2800/046; G01N 2800/36

(57) ABSTRACT

The discovery presents the means for confident diagnosis of chronic inflammatory disease of ovaries such as PID. The hormone producing function of ovaries is one of the main functions of them and if that function is disrupted, it could have the meaning that present is the condition of ovaries that requires treatment.

The thyroid anti-body disease is the nothing else as the sygnal of the endocrine system that currently there is present the serious inflammatory or other type of condition of ovaries which is requires the treatment. The any medical condition of ovaries that does the disruption of hormone producing function of them is the serious disease that requires the addressing.

The thyroid tissue is made up of two types of cells: follicular cells and parafollicular cells. The thyroid plays an important role in endocrine system: it regulates the body's metabolism and calcium balance that are controlled by T4 and T3 hormones. Level of hormones secreted by the thyroid are controlled by the pituitary gland's thyroid-stimulating hormone.

The levels of estrogen and progesterone that are produced by women's ovaries is controlled by gonadotropic hormones produced as well by pituitary gland.

So, the pituitary gland produces hormones for both of these systems: thyroid gland and women's ovaries.

As any taken system, the endocrine system should be balanced at most of the time. The reason for that, that the hormones produced should strictly correspond to the amount of all active cells in organism that they interact with or the active and healthy cells would not get the part of hormonal stimulation to perform theirs wide range of actions. That is why the gonadotropic and thyroid-stimulating hormone are produced at the same place: pituitary gland. As those hormones produced by one single organ, we could confidently assume that theirs levels do correspond between themselves (at the other words, the amounts of the above listed hormones are interconnected): the how much gonadotropic hormones were produced, the corresponding amount of thyroid-stimulating hormones would be produced in pituitary gland.

Now, about thyroid anti-body disease. At the case when there is chronic or possible malignant inflammation of ovaries, the ovaries don't secret enough hormones. But the produced amount of estrogen and progesterone is controlled by gonadotropic hormone, and so the amount of that hormone would exceed the needs of sick ovaries. The endocrine system needs to turn on the “manual mechanism” of override and reduce the secretion of gonadotropic hormone. The only way to lower the production of gonadotropic hormone would be first to adjust (to destroy) the some of thyroid cells that are producing T4 and T3 hormones; then as the second the amount of thyroid-stimulating hormone which is produced by pituitary gland would be lowered too (there is the back-forth connection between them); and it, at the third, in own turn, would reduce the amount of gonadotropic hormone to correspond to the thyroid-stimulating hormone, what was needed.

The treatment of thyroid anti-body disease should consist at the addressing the problem with hormone secreting functions of ovaries. The hormone secreting function of ovaries could be a result of chronic PID (pelvic inflammation disease) or, may be, malignancy of ovaries or connected tissues? At the case of PID, the antibiotics or other appropriate treatment should be applied. The thyroid anti-body disease would be cured by itself the after the PID would be cured.

### BACKGROUND OF INVENTION

The study and analysis were performed by the author of invention just on the one clinical case of thyroid anti-body disease. The author was studying the own case of illness and treatments applied and came to this revolutionary discovery. The medical records applying to that case could be found at Sutter Gould Medical Foundation for 2010-2011 years.

### SUMMARY

There was a belief that the reason of thyroid anti-body disease is mistaken identity. That discovery revolutionizes the general outlook on the functioning of endocrine system and also gives the detailed explanation of mechanism of thyroid anti-body disease and possible treatment of that. The currently there is no treatment for this disease is offered.

### DESCRIPTION

The patient was having the long, not diagnosed serious problem with gynecology. The after, approximately, half of the year after that problem, the thyroid anti-body disease started as the patient had noticed the changes in thyroid. The some time after that, the inflammatory gynecological disease was fouind and the treatment was applied. The thyroid anti-body disease was cured.

The medical personnel of Sutter Gould Medical Foundation was not aware the what was going on. The author of invention did analysed the results of tests and treatment and came to the conclusion that the thyroid anti-body disease at her case was the result of long not treated inflammatory disease of ovaries.

The author believes that the some of the cases of chronic inflammatory diseases could trigger the thyroid anti-body disease as the response of endocrine system to the reduction of produced estrogen and progesteron. The author believes if there is suspected PID or other chronic infection of ovaries, there should be performed the test on the presence of thyroid anti-body cells: at the order to confirm the diagnosis of suspected chronic infection which is characterised by disruption in the ovaries hormone producing function. If the such test would show the presence of thyroid anti-body cells, then the disease would be confirmed and it could be addressed.

1. Thyroid anti-body disease at some of the cases is the results of inflammatory diseases of ovaries which is characterised by disruption in theirs function of hormone secretion; that could be the PID or other chronic infection.

2. The treatment of thyroid anti-body disease should consist from addressing the underlying illness of women's ovaries.

3. To confirm the diseases of ovaries which may be characterised by the disruption of hormone secretion function, need to perform the test for presence of thyroid anti-body cells.
